(19) United States
(12) Plant Patent Application Publication
(10) Pub. No.: US 2007/0234456 P1
(43) Pub. Date: Oct. 4, 2007

(54) IMPATIENS PLANT NAMED 'TIROSE'

(76) Inventors: Harlan B. Cosner, Rogue River, OR (US); Susan Cosner, Rogue River, OR (US)

Correspondence Address: GANZ LAW, P.C., P O BOX 2200, HILLSBORO, OR, 97123, US

(21) Appl. No.: 11/393,388

(22) Filed: Mar. 30, 2006

(51) Int. Cl. A01H 5/00

(52) U.S. Cl. PLT/319

(57) ABSTRACT

A new double Impatiens walleriana producing rose colored flowers; dark green foliage; fully double flowers held above or beyond the foliage on strong peduncles and pedicels; and a prostrate mounded growth habit.

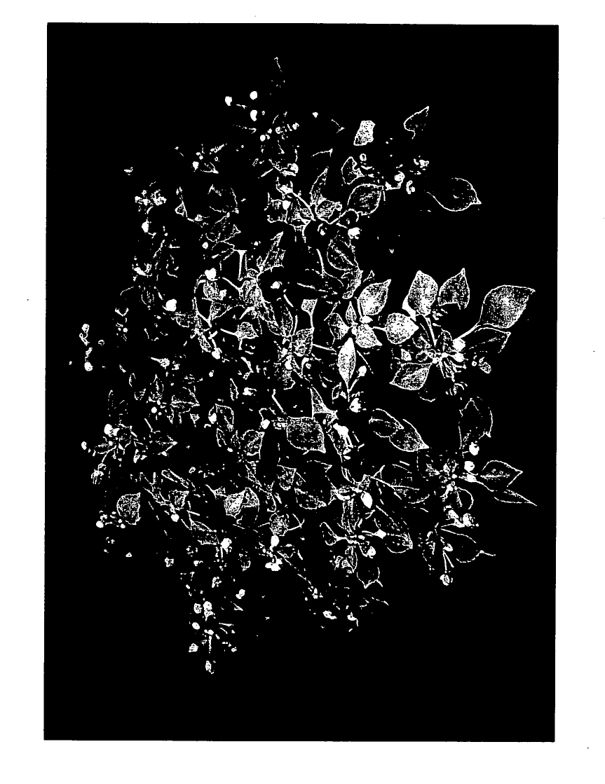

Latin name of the genus and species and variety denomination: Impatiens walletiana plant and known by the cultivar name ‘TiRose.’

### BACKGROUND OF THE INVENTION

1. Field of the Invention

The present invention comprises a new and distinct cultivar of plant, botanically known as Impatiens walleriana, and hereinafter referred to by the cultivar name of ‘TiRose.’
1. The plants are useful in landscaping, window and hanging baskets and flower gardens.
2. The plant is a double flowered Impatiens Walleriana.
3. The plants are generally classed as an annual bedding plant.

2. Description of the Relevant Prior Art

1. The pollen parent is an unpatented pollen producing double Impatiens walleriana producing purple-colored flowers (numbered M-BR-13) and the seed parent is an unpatented semi-double Impatiens walleriana producing hot pink-colored flowers (numbered M-BR-127). (The cross was made on Oct. 11, 2001; the seed was planted on Jan. 12, 2002. and the first asexual reproduction was made on Jun. 18, 2002).

2. The seed parent was hot pink and the pollen parent was purple, neither parent is patented nor commercially available. The cross was made in the breeder's controlled breeding program at Rogue River, Oreg. The first asexual reproduction was also made at Rogue River, Oreg. Successive asexual generations produced from cuttings with leaves have shown the cultivar to be stable.

### COMPARISON

1. The plant is compared to ‘TiHop’, U.S. Plant Pat. No. 13,038.
2. The flower color of ‘TiHop’ is lighter than the color of ‘TiRose.’
3. The plant of TiHop is larger than the plant of ‘TiRose’
4. See comparison chart.

### SUMMARY OF THE INVENTION

The cultivar is unique either in one or a combination of the characteristics described herein It is a new, double Impatiens walleriana producing rose flowers; dark green foliage; fully double flowers held above or beyond the foliage on strong peduncles and pedicels; and a mounded growth habit.

### BRIEF DESCRIPTION OF THE DRAWING

The attached color photographs illustrate a plant of ‘TiRose’ grown at Rogue River, Oreg. and flowered under winter low light conditions. The photograph was taken on or about Feb. 21, 2006. Variations between the written description and the colors in the photographs may occur due to the type of film, the light reflectance and the type of light captured by the film, the winter conditions under which the plant in the photograph was grown, and/or the summer high light conditions under which the plant described in the “DETAILED BOTANICAL DESCRIPTION” that follows. In the event there are such variations, the following written description shall control.

### DETAILED BOTANICAL DESCRIPTION

The following description was taken at Rogue River, Oreg. on Aug. 17, 2005. The description is of a plant of ‘TiRose’ similar to the one shown in the photograph. The accompanying color photograph was taken of a ‘TiRose’ plant of about 10 weeks of age from rooted cuttings at first inflorescence and illustrates a plant of ‘TiRose’ grown in summer and just beginning to mature.

Color references are according to The Royal Horticultural Society Chart, except where general terms of ordinary dictionary significance are used.

The following description was taken under the following environment and cultural practices at Rogue River, Oreg. Phenotypical variations may result in plants grown under conditions and locations other than those listed herein.
Container.—6 inch azalea pot.
Medium.—Peat-lite type of soilless medium.
Greenhouse covering.—Double layer Polyethelene with 50% shade applied above the covering.
Minimum temperature.—70° F.
Maximum temperature.—105° F.
Light levels.—2500 ft candles.
Fertilizer.—20-10-20 with trace elements applied twice followed by one leaching with clear water.
Cutting type.—Lateral stems with leaves.
Propagation:
   Type of cutting.—Lateral stems with leaves.
   Time to initiate roots.—Generally about 7 to 10 days at soil temperature of 72 degrees F.
Appearance and form of mature plant:
   Growth habit.—Prostrate Mounded.
   Plant size.—Plants mature, at about 15 cm in height and about 20 cm in width. Both of these measurements are a function of age, environmental and cultural practices, and can vary accordingly.
   Rooting habit.—The roots are fibrous and well branched.
   Rooting ability.—Easy, no hormones needed.
   Stems.—Stems are freely produced and no pinching to induce branching is needed. Color is close to 146D with a dark spot at the node where the petiole attaches that is close to 146B. The stems at maturity are generally about 0.4 cm in diameter at the internodes, and the internode length is generally about 1.75 cm in length. The stem length at maturity is about 10 cm.
   Foliage.—Shape is ovate with cuneate to attenuate base; crenate margin, margin having tiny spines of an undeterminable greenish color and each being about 1.0 mm in length and protruding outwards from leaf edge at the lobes of the crenate margin and parallel to the flat surfaces of the leaf, and an acuminate apex. Size of largest is about 6.0 cm long; about 3.0 cm wide.
   Foliage color.—Adaxial surface close to 146A; abaxial surface close to 148B; abaxial surface venation is close to 146A and is easily distinguishable; adaxial surface venation is closest to 146A and is difficult to determine.
   Foliage texture.—Smooth and flat.
   Petioles.—Longest petioles are about 3 cm long, half round, flat to slightly convex on upper surface which is about 3 mm wide, thickness (from the upper surface to the bottom surface) of about 2 mm. Adaxial surface color appears to be closest to 146A; abaxial surface color appears to be closest to 146D.
   Flower habit.—Flowers are produced continuously throughout the flowering season.
   Natural flowering season.—Year around in greenhouse conditions, and the frost free period from ring through fall when planted outdoors.
   Duration of flower.—Each flower generally lasts about 4 days at 115° F. maximum and 66° F. minimum temperature.
   Fragrance of flower.—No fragrance observed.
   Flowers borne.—Above or beyond the foliage.
   Flower texture.—Smooth and satiny.
   Flower color.—Petals/petaloids — Adaxial surface is close to 66A some have a dark streak in the center that is close to 61B. Abaxial surface is close to 63D.
   Quantity.—Usually 2 or more open flowers per stem and generally 12 or more from visible buds to fully mature flowers.
   Petal/petaloid count.—Generally 20 or more.
   Size.—Flowers are generally at least 3.0 cm in diameter; generally at least 2.0 cm deep. The largest petals/petaloids are generally at about 2.0 cm wide, and about 1.5 cm long; obovate to exaggerated obovate in shape with obtuse to retuse apex, entire margin, and cuneate to obtuse base. The above measurements refer to the flowers as measured during prolonged heat with daily maximums between 105° F. and 115° F. Flowers may be larger in cooler temperatures.
   Flower buds.—The buds prior to opening are about 0.7 cm in length; about 0.7 cm in diameter, shape is ovate. Bud color prior to opening is close to 145B on top and close to 145D on bottom.
   Peduncles.—Generally about 3.0 cm long; about 2.5 mm in diameter; color close to 144B. Good strength.
   Pedicels.—Generally two or more per peduncle. Each is generally about 2.75 cm in length, and about 2.0 mm in diameter. Color is close to 144B. Good strength.
   Calyx.—Calyx consists of one sepal with one spur attached near the base.

The spur is generally at least 2.5 cm long, and about 1.0 mm in diameter at sepal end; shape is an acicular tapering tube generally curved, and it is attached toward the base of the sepal in a peltate manner. Color is close to 144A at apex, changing to 153B and close to 152C at the base.

The sepal generally measures about 0.6 cm wide, and about 1.0 cm long; ovate in shape with an acute to cuspidate apex, entire margin, and obtuse to cordate base; adaxial surface is close to 145B at the apex, and the base is close to 145C with a tiny reddish spot around where the spur connects that is too small to accurately be determined but appears close to a greenish tinged 67A; the abaxial surface is between 145B, with an apex color close to 144A.
Reproductive organs.—The reproductive organs are replaced with sterile petaloids.
Disease resistance.—Disease resistance has not been tested.
Dampness resistance.—The plant has shown a good ability to hold the flowers in an outward mariner during summer rains.

Heat tolerance.—The plant has exhibited excellent heat tolerance in garden and greenhouse trials. It both grows and flowers uninterrupted during prolonged periods with maximum temperatures above 90° F. and at least as high as 115° F. with Relative Humidity at least as low as 20% and in Greenhouse temperatures as high as 120° F. without any adverse effects, such as reverting to vegetative state, decreased flower size, yellowing of new growth, bud drop, or root kill.

TABLE 1
COMPARISON CHART TO PRIOR ART

| | ‘TiRose’ | ‘TiHop’ |
|---|---|---|
| Stems-Color | 146D in internodes, darker at | 146A with dark markings |
| | nodes close to 146B | close to 183A |
| Foliage-Size-Length | About 6 cm | About 7 cm |
| Width | About 3 cm | About 4.5 cm |
| Foliage-Abaxial venation | Close to 146A | Close to 148A |
| color | | |
| Foliage-Adaxial color | Close to 146A | Darker than 147A |
| Abaxial color | Close to 148B | 146B developing darker |
| | | markings with age close to |
| | | 177A |
| Foliage-Adaxial Venation | Closest to 146A | 147A |
| Color | | |
| Petiole-Size | About 3 cm long, upper | About 4.5 cm long and about |
| | surface which is about 3 mm | 4 mm width, thickness about |
| | wide, thickness of about 2 mm | 2 mm |
| Adaxial color | Closest to 146A | 147B |
| Petiole-Abaxial color | Closest to 146D | Close to 174C at base, |
| | | darkening to 147B at leaf end |
| Petal/Petaloid-Size-Width | About 2 cm | About 2 cm |
| Length | About 1.5 cm | About 2.5 cm |
| Abaxial color | Close to 63D | Close to 66D |
| Petal/Petaloid Adaxial color | 66A, some have a streak close | Close to 66A, with a base spot |
| | to 61B | close to 59D; some have a |
| | | stripe close to 61B |
| Peduncle-Size-Length | About 3 cm | About 3 cm |
| Diameter | About 2.5 mm | About 2 mm |
| Color | Close to 144B | 146B with purplish overtones |
| Pedicel-Size-Length | About 2.75 cm | About 2.5 cm |
| Diameter | About 2 mm | About 1.5 mm |
| Color | Close to 144B | Color is 146B to 146C with |
| | | reddish purple overtones |
| Sepal-Size-Length | About 1 cm | About 1.2 cm |
| Width | About .6 cm | About 0.9 cm |
| Adaxial color | close to145B at apex | 145D with dark base close to |
| | changing to 145C at base | 61A, with spots toward |
| | | margin 61A with a dark apex |
| | | of undeterminable color |
| Sepal Abaxial color | Close to 145B with apex close | 145D with markings close to |
| | to144A | 66B and dark apex of |
| | | undeterminable color |
| Spur-Size-Length | About 2.5 cm | About 3 cm |
| Diameter at base | About 1 mm | About 2 mm |
| Color | Apex close to144A changing | Close to 181B changing |
| | to close to 153B in mid- | toward 187B at base and |
| | section and close to 152C at | toward 181C at apex |
| | base | |
| Buds-Size-length | About 0.7 cm | About 1 cm |
| Diameter | About 0.7 cm | About 1 cm |
| Color | Close to 145B on top and | 143C on top and 145B on |
| | close to 145D on bottom | bottom |

COLOR CODE CHART
Color reference acording to the Royal Horticultural Society Chart

| | |
|---|---|
| Stems: | 146D, 146B |
| Foliage Abaxial surface: | 148B |
| Foliage Abaxial surface venation: | 146A |
| Foliage Adaxial surface: | 146A |
| Foliage Adaxial surface venation: | 146A |
| Petiole Abaxial surface: | 146D |
| Petiole Adaxial surface: | 146A |
| Petals/Petaloids Abaxial surface: | 63D |
| Petals/Petaloids Adaxial surface: | 66A, 61B |
| Buds: | 145B, 145D |
| Peduncles: | 144B |
| Pedicels: | 144B |
| Sepals Adaxial surface: | 145B, 145C |
| Sepals Abaxial surface: | 145B, 144A |
| Spur: | 144A, 153B, 152C |

It is claimed:

1. A new and distinct cultivar of double Impatiens walleriana plant named ‘TiRose,’ as illustrated and described herein.
